(19) United States
(12) Patent Application Publication
(10) Pub. No.: US 2026/0053554 A1
(43) Pub. Date: Feb. 26, 2026

(54) BIPOLAR TISSUE VAPORIZATION DEVICE WITH NON-VAPORIZING SURFACE

(71) Applicant: Boston Scientific Scimed, Inc., Maple Grove, MN (US)

(72) Inventors: Christian BALKOVEC, Cambridge (CA); Gareth DAVIES, Toronto (CA)

(21) Appl. No.: 19/271,079

(22) Filed: Jul. 16, 2025

Related U.S. Application Data
(60) Provisional application No. 63/685,662, filed on Aug. 21, 2024.

(51) Int. Cl. A61B 18/14; A61B 18/12; A61B 18/00; A61B 18/16

(52) U.S. Cl. CPC A61B 18/1492; A61B 18/1206; A61B 2018/00357; A61B 2018/00625; A61B 2018/162

(57) ABSTRACT

An electrosurgical system for removing tissue includes an electrosurgical generator configured to generate radiofrequency (RF) energy, and a crossing device connected to the electrosurgical generator. The crossing device includes a first electrode configured to deliver the RF energy to the tissue from the electrosurgical generator and a second electrode configured to return the RF energy to the electrosurgical generator. The first electrode is configured to form an electrically insulative gaseous layer when delivering the RF energy.

### CROSS REFERENCE TO RELATED APPLICATIONS

This application claims priority to U.S. Provisional Patent Application No. 63/685,662 entitled “BIPOLAR TISSUE VAPORIZATION DEVICE WITH NON-VAPORIZING SURFACE,” filed August 21, 2024, which is hereby incorporated by reference in its entirety.

### TECHNICAL FIELD

The present disclosure relates to medical systems and methods for vaporizing tissue in a patient. More specifically, the present disclosure relates to medical systems and methods for puncturing and slicing bodily tissues such as the atrial septum with an electrode by dielectric breakdown.

### BACKGROUND

Minimally invasive surgical techniques can employ catheters to provide access into a patient’s body. Surgical instruments can be routed through the catheters such that the instruments can be positioned where they are needed for a given procedure. For example, in a cardiac procedure involving the left atrium of the heart, it may be advantageous to route a catheter into the right atrium and perform a transseptal puncture procedure to enter the left atrium. Such a transseptal puncture procedure can be performed using one or more electrodes that are passed through a catheter and energized in the radiofrequency (RF) range. While this procedure can vaporize the targeted septal tissue, it can also vaporize liquids (e.g., blood) that are in contact with the electrode(s).

### SUMMARY

In an Example 1, an electrosurgical system for removing tissue, the electrosurgical system comprising: an electrosurgical generator configured to generate radiofrequency (RF) energy; and a crossing device connected to the electrosurgical generator, the crossing device including a first electrode configured to deliver the RF energy to the tissue from the electrosurgical generator and a second electrode configured to return the RF energy to the electrosurgical generator; wherein the first electrode is configured to form an electrically insulative gaseous layer when delivering the RF energy.

In an Example 2, the electrosurgical system of Example 1, wherein the second electrode is configured to not form an electrically insulative gaseous layer when returning the RF energy.

In an Example 3, the electrosurgical system of Examples 1 or 2, wherein the first electrode has a smaller surface area than the second electrode.

In an Example 4, the electrosurgical system of any of Examples 1-3, wherein the first electrode has a rougher surface finish than the second electrode.

In an Example 5, the electrosurgical system of any of Examples 1-4, wherein the first electrode is shorter than the second electrode.

In an Example 6, the electrosurgical system of any of Examples 1-5, wherein the first electrode is positioned at a distal end of the crossing device, and the second electrode is positioned proximal from the first electrode.

In an Example 7, the electrosurgical system of Example 6, wherein the first electrode includes a sharpened distal tip.

In an Example 8, the electrosurgical system of any of Examples 1-5, wherein the second electrode is positioned at a distal end of the crossing device, and the first electrode is positioned proximal from the second electrode.

In an Example 9, the electrosurgical system of Example 8, wherein the second electrode includes a sharpened distal tip.

In an Example 10, the electrosurgical system of any of Examples 1-5, wherein the first electrode and the second electrode are positioned proximal from a distal end of the crossing device.

In an Example 11, the electrosurgical system of Example 10, wherein the crossing device includes a sharpened distal tip.

In an Example 12, the electrosurgical system of any of Examples 1-11, wherein the crossing device includes a central lumen.

In an Example 13, the electrosurgical system of Example 12, further comprising an active conductor electrically connected to the first electrode.

In an Example 14, the electrosurgical system of Example 13, further comprising a return conductor electrically connected to the second electrode.

In an Example 15, the electrosurgical system of Example 14, wherein: the active conductor extends through a first offset lumen; the return conductor extends through a second offset lumen; and the first and second offset lumens extend alongside the central lumen.

In an Example 16, an electrosurgical system for removing tissue, the electrosurgical system comprising: an electrosurgical generator configured to generate radiofrequency (RF) energy; and a crossing device connected to the electrosurgical generator, the crossing device including a first electrode configured to deliver the RF energy to the tissue from the electrosurgical generator and a second electrode configured to return the RF energy to the electrosurgical generator; wherein the first electrode is configured to form an electrically insulative gaseous layer when delivering the RF energy.

In an Example 17, The electrosurgical system of Example 16, wherein the second electrode is configured to not form an electrically insulative gaseous layer when returning the RF energy.

In an Example 18, The electrosurgical system of Example 16, wherein the first electrode has a smaller surface area than the second electrode.

In an Example 19, the electrosurgical system of Example 16, wherein the first electrode has a rougher surface finish than the second electrode.

In an Example 20, the electrosurgical system of Example 16, wherein the first electrode is shorter than the second electrode.

In an Example 21, the electrosurgical system of Example 16, wherein the first electrode is positioned at a distal end of the crossing device, and the second electrode is positioned proximal from the first electrode.

In an Example 22, the electrosurgical system of Example 16, wherein the second electrode is positioned at a distal end of the crossing device, and the first electrode is positioned proximal from the second electrode.

In an Example 23, the electrosurgical system of Example 16, wherein the first electrode and the second electrode are positioned proximal from a distal end of the crossing device.

In an Example 24, the electrosurgical system of Example 16, wherein the crossing device includes a central lumen.

In an Example 25, an electrosurgical system for puncturing tissue, the electrosurgical system comprising: an electrosurgical generator configured to generate radiofrequency (RF) energy; and a crossing system comprising: a steerable sheath;

a dilator slidably positioned in the steerable sheath; and a crossing device connected to the electrosurgical generator, the crossing device including a vaporizing electrode positioned at a distal end of the crossing device and a nonvaporizing electrode positioned proximal from the vaporizing electrode; wherein the vaporizing electrode is configured to deliver the RF energy to the tissue from the electrosurgical generator and the non-vaporizing electrode is configured to return the RF energy to the electrosurgical generator.

In an Example 26, the electrosurgical system of Example 25, wherein the vaporizing electrode is configured to form an electrically insulative gaseous layer when delivering the RF energy.

In an Example 27, the electrosurgical system of Example 25, wherein the non-vaporizing electrode is configured to not form an electrically insulative gaseous layer when returning the RF energy.

In an Example 28, the electrosurgical system of Example 25, wherein the first electrode has a smaller surface area than the second electrode.

In an Example 29, the electrosurgical system of Example 25, wherein the first electrode has a rougher surface finish than the second electrode.

In an Example 30, the electrosurgical system of Example 25, wherein the first electrode is shorter than the second electrode.

In an Example 31, the electrosurgical system of Example 25, wherein the first electrode is positioned at a distal end of the crossing device, and the second electrode is positioned proximal from the first electrode.

In an Example 32, the electrosurgical system of Example 25, wherein the second electrode is positioned at a distal end of the crossing device, and the first electrode is positioned proximal from the second electrode.

In an Example 33, an electrosurgical system for slicing tissue, the electrosurgical system comprising: an electrosurgical generator configured to generate radiofrequency (RF) energy; and a crossing system comprising: a steerable sheath; a dilator slidably positioned in the steerable sheath; and a crossing device slidably positioned in the dilator and electrically connected to the electrosurgical generator, the crossing device including a sharpened distal tip, a vaporizing electrode positioned proximal from the distal tip, and a non-vaporizing electrode positioned proximal from the distal tip; wherein the vaporizing electrode is configured to deliver the RF energy to the tissue from the electrosurgical generator and the non-vaporizing electrode is configured to return the RF energy to the electrosurgical generator.

In an Example 34, the electrosurgical system of Example 33, wherein the vaporizing electrode is configured to form an electrically insulative gaseous layer when delivering the RF energy.

In an Example 35, the electrosurgical system of Example 33, wherein the non-vaporizing electrode is configured to not form an electrically insulative gaseous layer when returning the RF energy.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram illustrating an example electrosurgical system for treating a patient, such as a heart or the vasculature of a patient, including a bipolar electrosurgical generator and a transseptal crossing system, consistent with various aspects of the present disclosure.

FIG. 2 is a cutaway view of a heart including an atrial septum and the transseptal crossing system, consistent with various aspects of the present disclosure.

FIGS. 3A and 3B are views of a distal end of the transseptal crossing system. More specifically, FIG. 3A is a side view of the distal end, consistent with various aspects of the present disclosure. FIG. 3B is an exploded perspective view of a bipolar crossing device, consistent with various aspects of the present disclosure.

FIGS. 4A-4C are side views of alternative distal ends. More specifically, FIG. 4A shows a distal end with a roughened electrode, consistent with various aspects of the present disclosure. FIG. 4B shows a distal end with a corrugated electrode, consistent with various aspects of the present disclosure. FIG. 4C shows a distal end with a coiled electrode, consistent with various aspects of the present disclosure.

FIG. 5 is a flowchart of a method of crossing the atrial septum, consistent with various aspects of the present disclosure.

FIGS. 6A and 6B are a series of schematic diagrams of operations of crossing the atrial septum according to the method of FIG. 6, consistent with various aspects of the present disclosure.

FIG. 7 is a flowchart of a method of operating the electrosurgical system to puncture tissue, consistent with various aspects of the present disclosure.

FIG. 8A-8F are a series of schematic diagrams of operations of puncturing tissue according to the method of FIG. 8, consistent with various aspects of the present disclosure.

FIG. 9 is a flowchart of an alternative method of operating an alternative electrosurgical system to puncture and slice tissue, consistent with various aspects of the present disclosure.

FIGS. 10A and 10B are views of a distal end of a dissecting system. More specifically, FIG. 10A is a side view of the distal end, consistent with various aspects of the present disclosure. FIG. 10B is a cross-sectional view of the distal end as indicated by line 10-10 in FIG. 10A, consistent with various aspects of the present disclosure.

FIG. 11 is a side view of an alternative distal end of the dissecting system, consistent with various aspects of the present disclosure.

FIG. 12 is a flowchart of a method of operating the electrosurgical system to puncture and slice tissue, consistent with various aspects of the present disclosure.

FIG. 13A-13D are a series of schematic diagrams of operations of puncturing and slicing tissue according to the method of FIG. 12, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

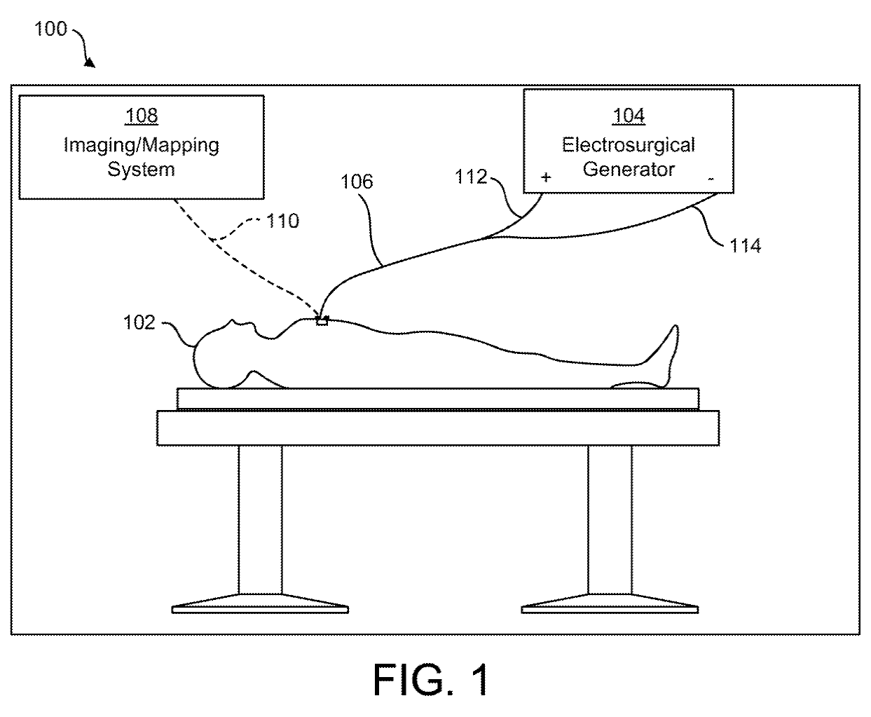

FIG. 1 shows an electrosurgical system 100 for treating a patient 102. In the illustrated embodiment, the system 100 includes a bipolar electrosurgical generator 104 with a transseptal crossing system 106 and an imaging/mapping system 108 for tracking the crossing system 106 in the patient 102. The imaging/mapping system 108 can use an external fluoroscopy system (not shown) and/or a mapping catheter 110 (shown in phantom) (such as, for example, the RHYTHMIA HDx^{TM} mapping system from the Boston Scientific Corporation).

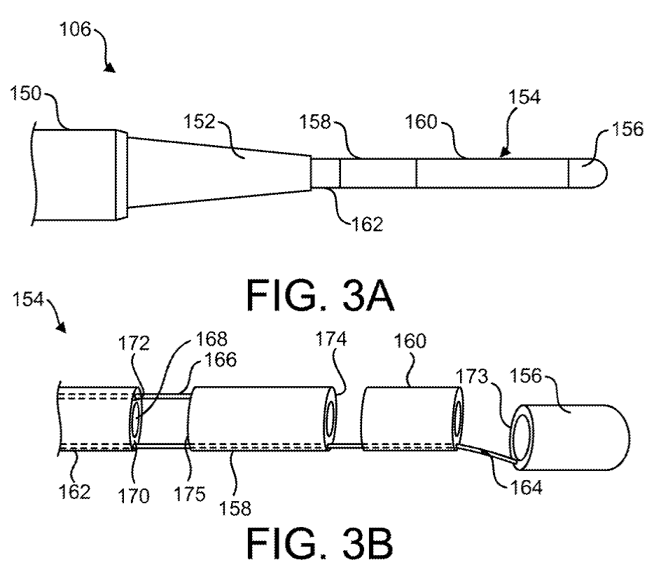

In the illustrated embodiment, the electrosurgical generator 104 is configured to provide energy, such as radiofrequency (RF) electrical energy, to the crossing system 106. The crossing system 106 is bipolar, so the crossing system 106 includes a positive lead 112 and a negative lead 114 that plug into the positive and negative terminals on the electrosurgical generator 104, respectively. The conductive portions of the crossing system 106 are electrically insulated with the exception of two small distal portions. One such portion is the “active” or vaporizing electrode, and the other such portion is the “return” or non-vaporizing electrode (both shown in FIG. 3A). These electrodes are configured to deliver and return the RF energy to and from the target tissue, respectively. To achieve tissue vaporization, the target tissue is rapidly heated by the vaporizing electrode. If heating is too slow, the tissue is desiccated rather than vaporized. Rapid heating is achieved through high current density at the vaporizing electrode-tissue interface.

The delivery of energy to the target tissue can also heat the crossing system 106 itself. Thus, the electrical insulation around the crossing system 106 (especially near the electrode) should be able to withstand such heat without breaking down. While typically the insulation has been made from per- and polyfluoroalkyl substances (PFAS) (e.g., polytetrafluoroethylene (PTFE)), other materials may have the advantage of being less environmentally problematic. Such alternative materials, however, may lack the heat performance of the traditional materials, so the crossing system 106 should be designed to reduce the heat generation within itself while still providing adequate heat generation in the target tissue.

In the illustrated embodiment of FIG. 1, the RF energy for a puncture function is provided by the electrosurgical generator 104 at a selected voltage and a continuous current (100% on, or 100% duty cycle). For example, if a power setting of 50 watts (W) is used for puncturing (which can mean that the instantaneous power is higher than 50 W), the voltage can range from approximately 164 volts (V) to 400 V root mean square (RMS). In addition, the electrosurgical generator 104 can include a plurality of functions and provide programmed and custom settings via an interface (not shown). For example, the electrosurgical generator 104 provides RF energy to the crossing system 106 as an alternating current having a frequency in the range of 100 kilohertz (kHz) to 10 megahertz (MHz). Such puncturing RF energy can be applied in the form of a continuous waveform signal or in bursts of a waveform signal. In the latter case, the individual bursts of the waveform signal can have a duration of about 300 milliseconds (ms) with a rest interval between pulses of about 700 ms, although other durations of bursts and intervals can be used. In some embodiments, the waveform signals themselves can be sinusoidal or square waves that are bi-phasic. Furthermore, the electrosurgical generator 104 can be couplable to other electrosurgical tools and/or the electrosurgical generator 104 can receive signals (e.g., from the crossing system 106) to monitor the patient 102.

The components and configuration of electrosurgical system 100 allow for target tissue to be vaporized without requiring a separate patch electrode (i.e., an indifferent or dispersive electrode) to be placed on the patient due to the bipolar nature of the crossing system 106. Thereby, such a patch electrode does not need to be included in the surgical equipment kit. In some embodiments, the crossing system 106 is configured to puncture through the atrial septum for treatment of the left side of the heart of the patient 102. In other embodiments, the crossing system 106 is configured to puncture and slice through a heart valve for modification of a valve leaflet. While examples of the devices, systems, and methods of the present disclosure are presented in the context of a transeptal puncture or leaflet modification, a person having ordinary skill in the art will recognize other applicable contexts. For example, the electrosurgical systems of the present disclosure can be employed to puncture a pericardium layer of a patient for epicardial access and/or to remove accumulation of atheromatous material on the inner walls of vascular lumens.

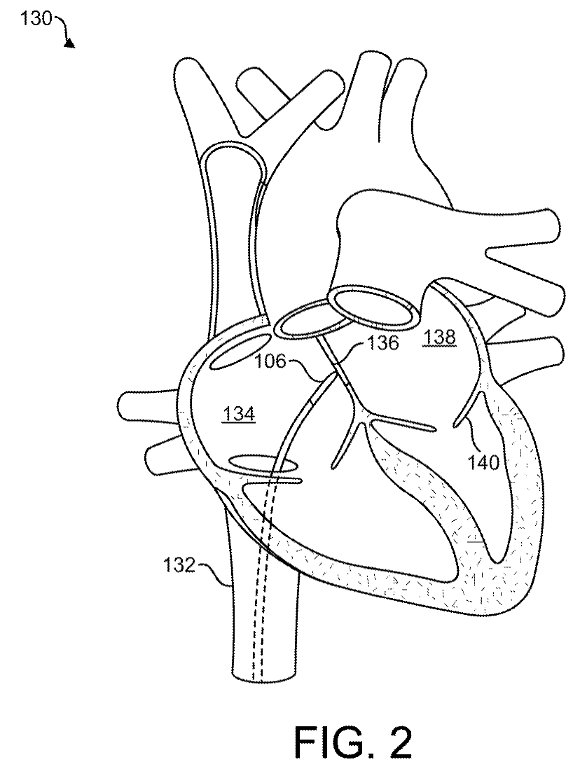

FIG. 2 shows a heart 130 of the patient 102 (shown in FIG. 1) with selected portions cut away. In the illustrated embodiment, the crossing system 106 extends through the inferior vena cava 132 from a surgical entry site (not shown) that is distal to the heart 130. The distal end of the crossing system 106 is positioned in the right atrium 134 and is in contact with the atrial septum 136. In the illustrated embodiment, the tip of the crossing system 106 is positioned at the fossa ovalis since this region of the atrial septum 136 is relatively safe and easy to puncture. Once the atrial septum 136 is crossed, the physician will have access to the left atrium 138 (e.g., for treatment thereof). In some embodiments, the leaflets of the mitral valve 140 can then be modified due to othe access to the left atrium 138.

In some use cases, such as a septal crossing, the electrode will be surrounded by a conductive liquid medium, such as blood, that is near the target tissue. When the crossing system 106 is operating in such an environment, any surface of the electrode that is in contact with the conductive liquid (instead of with the target tissue) provides a shunt path for the electrical current. These alternative electrical pathways do not help vaporize the target tissue, so they decrease the eﬃciency of the tissue vaporization process. Furthermore, these pathways can cause the blood to locally coagulate and form thrombotic material that can lead to embolization and subsequent deleterious health eﬀects for the patient 102 (shown in FIG. 1). Therefore, components and methods to prevent such occurrences are discussed in the present disclosure.

FIG. 3A shows a distal end of the crossing system 106. In the illustrated embodiment, the crossing system 106 includes a sheath 150, a dilator 152, and a bipolar crossing device 154. The crossing device 154 is an elongated tissue vaporization device that can have the form of, for example, a wire, a needle, forceps, scalpels, or other devices that puncture and/or cut tissue. In some embodiments, a wire is a solid, stiff but elastically deformable member with a generally straight and/or helical configuration. In some embodiments, a needle is a hollow, flexible member with a generally straight configuration through which fluid can be pumped. The fluid can exit near an electrode that is positioned at the distal end of the needle, and the electrode can be connected to the electrosurgical generator 104 with a conductor since the flexible member can be made from an electrically insulating material. In some embodiments, forceps are a dual-levered instrument capable of grasping and/or holding tissue or other objects between their distal ends. In some embodiments, a scalpel is a bladed instrument with a sharpened edge capable of cutting tissue or other objects.

In some embodiments, the crossing system 106 has an overall length between about 55 centimeters (cm) and 300 cm. The sheath 150 is an elongate member with a central lumen (not shown), in which the dilator 152 and the crossing device 154 are slidably positioned. The central lumen diameter is similar to the outer diameter of the majority of the dilator 152 (except for the distal tip), and the sheath 150 is tapered at the distal end to make the transition between the sheath 150 and the dilator 152 smoother. In addition, the sheath 150 can be a steerable sheath and/or have a fixed or adjustable curve at the distal end for positioning of the dilator 152 and the crossing device 154. In some embodiments, the sheath 150 and the dilator 152 are generally similar to those of the VersaCross^{TM} Access Solution from Boston Scientific.

In the illustrated embodiment, the dilator 152 is an elongate member with a central lumen (not shown), in which the crossing device 154 is slidably positioned. The central lumen diameter is similar to the outer diameter of the crossing device 154, and the dilator 152 is tapered at the distal end to make the transition between the dilator 152 and the crossing device 154 smoother. In addition, the dilator 152 can have a fixed or adjustable curve at the distal end, for example, for positioning of the crossing device 154 against the atrial septum 136 (shown in FIG. 2).

In the illustrated embodiment, the crossing device 154 is an elongate member with a vaporizing (“V”) electrode 156 positioned at the distal end and a non-vaporizing (“N”) electrode 158 positioned proximal therefrom. The electrodes 156, 158 are separated and electrically insulated from each other by an insulator 160. In addition, the N electrode 158 is connected to the distal end of a shaft 162, which can also be an electrically insulating component. The V electrode 156 is electrically connected to one of the leads 112, 114 (shown in FIG. 1), and the N electrode 158 is electrically connected to the other one of the leads 112, 114.

FIG. 3B shows an exploded view of the crossing device 154 including the V electrode 156, the N electrode 158, the insulator 160, the shaft 162, an active conductor 164, and a return conductor 166. In the illustrated embodiment, the crossing device 154 includes a central lumen 168 that is empty. In other embodiments, a non-conductive stiffening member (e.g., a polymer rod or a metallic rod covered with electrical insulation) is positioned in the central lumen 168. In some embodiments, a diameter of the crossing device 154 is between about 0.50 millimeters (mm) and about 1.0 mm.

In the illustrated embodiment, the active (“A”) conductor 164 is electrically connected to the V electrode 156, and the return (“R”) conductor 166 is electrically connected to the N electrode 158. The conductors 164, 166 extend through offset lumens 170, 172, respectively, that are positioned alongside of the central lumen 168. The conductors 164, 166 and the electrodes 156, 158 comprise electrically conductive material, such as, for example, stainless steel, nitinol, platinum, gold, iridium, or combinations thereof. The A conductor 164 extends through the N electrode 158, so the A conductor 164 includes an exterior electrically insulative layer. Such an electrically insulative layer, the insulator 160, and the shaft 162 comprise electrically insulative material, such as, for example, parylene, polyimide, polyethylene terephthalate (PET), polyurethane, silicon dioxide (SiO₂), PTFE heat shrink, or combinations thereof).

In the illustrated embodiment, the V electrode 156 has a hemispherical distal end and a cylindrical shape proximal therefrom that terminates with a circular proximal edge 173 where the proximal end of the V electrode 156 and the distal end of the insulator 160 are coterminous. Thus, the electrode 158 is atraumatic (i.e., not sharp). In other embodiments, however, the crossing device has a solely cylindrical distal end. In addition, the N electrode 158 has a cylindrical shape that terminates with circular proximal ends 174, 175 where the N electrode 158 meets the insulator 160 (at the distal end) and the shaft 162 (at the proximal end), respectively. In general, the V electrode 156 is configured to vaporize tissue when energized in an electrically conductive fluid environment (e.g., when surrounded by blood), while the N electrode 158 is configured to not vaporize tissue. To these ends, the surface area of the V electrode 156 is about 1.0 mm² to about 3.5 mm², and the surface area of the N electrode 158 is greater than about 3.5 mm². In some embodiments, the surface area of the N electrode 158 is at least about 1.5 times larger than that of the V electrode 156 or at least 3.0 times larger.

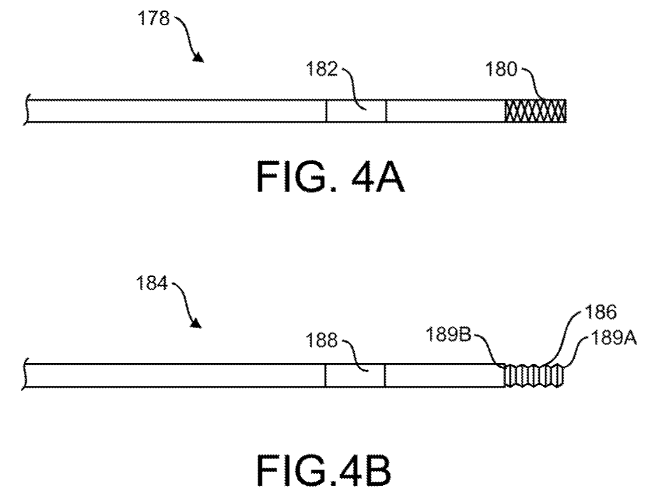

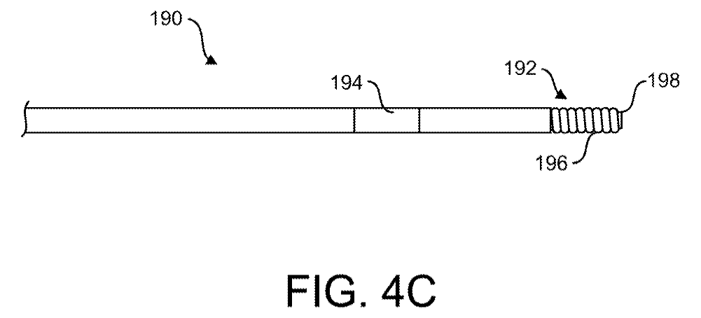

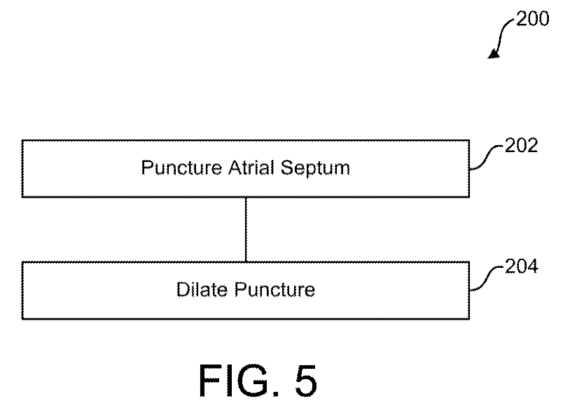

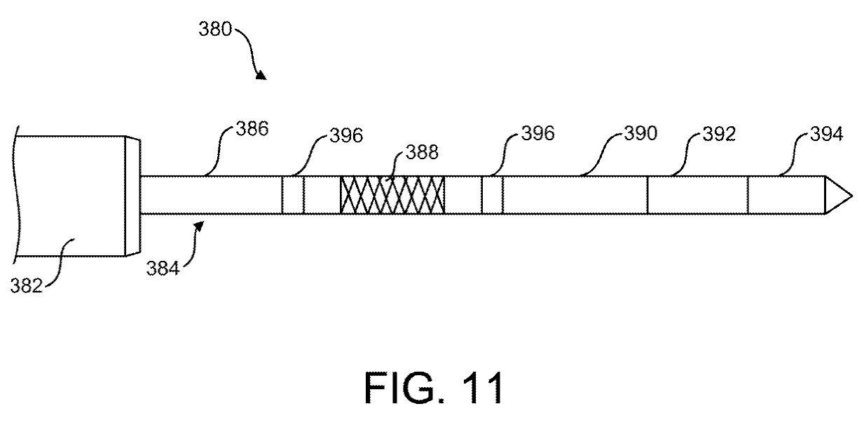

The mechanism by which the V electrode 156 vaporizes tissue while the N electrode 158 does not depend on gaseous bubble formation from the electrically conductive fluid and the retention of the gaseous bubbles. The formation and retention of bubbles can be affected by the geometric configurations of the electrodes 156, 158 and the differences therebetween. For example, a surface area differential between the electrodes 156, 158 can factor into differentiating the V electrode 156 from the N electrode 158, because a smaller surface area is more likely to form and retain bubbles than a larger surface area (as shown in FIG. 3A). For another example, a length differential between the electrodes 156, 158 can factor into differentiating the V electrode 156 from the N electrode 158, because a shorter length is more likely to form and retain bubbles than a longer length (as shown in FIG. 11A). For another example, a surface texture differential between the electrodes 156, 158 can factor into differentiating the V electrode 156 from the N electrode 158, because a rougher and/or contoured surface texture is more likely to form and retain bubbles than a flat and/or smooth surface texture (as shown in FIGS. 4 and 5). In some embodiments, a combination of such factors can be balanced appropriately to differentiate the V electrode 156 from the N electrode 158. In addition, which of the electrodes 156, 158 is vaporizing and which one is non-vaporizing is predetermined and fixed by the configurations thereof, so undesired tissue vaporization can be avoided (with respect to a situation where there is a lack of differentiation between the electrodes such that either electrode can be vaporizing).

FIG. 4A shows an alternative crossing device 178 wherein the V electrode 180 has a roughened surface texture that includes, for example, irregularities, etching, deformations, notches, or other features that result in a non-uniform and/or non-smooth surface. In some embodiments, the surface texture of the V electrode 180 has an arithmetic average roughness profile (Ra) between about 52 to about 64, between about 55 to about 61, or about 58. The surface texture of the V electrode 180 can be formed using any suitable method, for example, by removing material (e.g., using abrasive material (e.g., 240 grit sandpaper) or a chemical etching process) or by displacing/deforming material (e.g., using a knurling tool). Accordingly, the topography of the surface texture of the V electrode 180 can be random or it can be orderly with an orientation (e.g., longitudinal, circumferential, helical, or criss-cross).

In contrast, the N electrode 182 has a smooth surface texture. In some embodiments, the surface texture of the N electrode 182 has an Ra that is less than about 58, less than about 55, or less than about 52. The surface texture of the N electrode 182 can be formed using any suitable method, for example, by mechanical processes (e.g., polishing or melting) or by chemical processes (e.g., etching). In addition, the N electrode 182 can include other features that create a more hydrophilic surface on some or all of the N electrode 182 when compared to the surface of the V electrode 180.

In some embodiments, the size of the electrodes 180, 182 are substantially the same. In some embodiments, the diameter of the electrodes 180, 182 is about 0.50 millimeters (mm) to about 1.0 mm. In some embodiments, the length of the electrodes 180, 182 is about 0.50 mm to about 1.5 mm. In some embodiments, the exposed surface area of the electrodes 180, 182 is about 1.0 mm² to about 3.5 mm² each.

The rougher surface texture of the V electrode 180 provides irregularities that increase local electrical current density. Thus, the irregularities serve as nucleation sites for gaseous bubbles due to the local heating thereat. Furthermore, the irregularities allow the gaseous bubbles to cling to the V electrode 180 in a Wenzel state. Such a configuration is in contrast with the smooth surface texture of the N electrode 182, because the N electrode 182 lacks many of the nucleation sites that the V electrode 180 has. Thereby, the N electrode 182 will not generate or retain as many (or any) gaseous bubbles as the V electrode 180 does.

FIG. 4B shows an alternative embodiment of a crossing device 184 wherein a V electrode 186 has a corrugated surface texture. In the illustrated embodiment, the corrugations give the V electrode 186 an accordion-like shape due to an array of grooves formed in the V electrode 186. In some embodiments, the grooves are about 0.029 mm deep. The surface texture of the V electrode 186 can be formed using any suitable method, for example, by removing material (e.g., using a cutting tool) or by displacing material (e.g., a crimping or rolling die). In other embodiments, the surface texture is oriented in other directions, such as, for example, longitudinally or helically. In the former such embodiment, the electrode would have a splined shape due to the array of grooves being oriented longitudinally. In the latter such embodiment, the electrode would have a threaded shape with a single helical groove that winds around and along the length of the V electrode 186.

Regardless of the orientation, the peak(s) and/or valley(s) of the surface texture serve as nucleation sites for gaseous bubbles due to the locally increased current density and heating. The surface texture also allows the gaseous bubbles to cling to the V electrode 186 in a Wenzel state in the groove(s). Furthermore, the proximal and distal circular edges 189A-189B of the V electrode 186 also provide nucleation sites for gaseous bubbles. In other embodiments, the distal end of the V electrode is hemispherical or sharpened, so there would not be a distal circular edge. But in such embodiments, there would still be a proximal circular edge.

In contrast, the N electrode 188 has a smooth surface texture. In some embodiments, the surface texture of the N electrode 188 has an Ra that is less than about 58, less than about 55, or less than about 52. The surface texture of the N electrode 182 can be formed using any suitable method, for example, by mechanical processes (e.g., polishing) or by chemical processes (e.g., etching). In addition, the N electrode 188 can include other features that create a more hydrophilic surface on some or all of the N electrode 188 when compared to the surface of the V electrode 186.

In some embodiments, the size of the electrodes 186, 188 are about the same. In some embodiments, the diameter of the electrodes 186, 188 is about 0.50 millimeters (mm) to about 1.0 mm. In some embodiments, the length of the electrodes 186, 188 is about 0.50 mm to about 1.5 mm. In some embodiments, the exposed surface area of the electrodes 186, 188 is about 1.0 mm² to about 3.5 mm² each. Due to the corrugations of the V electrode 186, the V electrode 186 may be shorter than the N electrode 188 so that the electrodes 186, 188 have approximately the same exposed surface areas.

FIG. 4C shows an alternative embodiment of a crossing device 190 wherein a V electrode 192 is has a single helical groove that winds around the length of the V electrode 192. The groove is defined by an electrically conductive helical coil 196, and the coil 196 is wrapped around an electrically conductive mandrel 188. The diameter of the coil 196 is about 0.058 mm and the diameter of the mandrel 198 is about 0.50 mm, so the diameter of the coil 196 is about one tenth of the diameter of the mandrel 198. In some embodiments, the coil 196 is welded, soldered, brazed, adhered, or otherwise affixed to the mandrel 198 such that the coil 196 can transmit electricity to the patient 102 (shown in FIG. 1). The coil 196 can comprise the same or different material from that of the mandrel 198. Thus, the peak of the surface texture (i.e., the outermost portion of the coil 196) provide nucleation sites for gaseous bubbles due to the locally increased current density and heating. The surface texture also allows the gaseous bubbles to cling to the V electrode 192 in a Wenzel state in the groove.

In contrast, the N electrode 194 has a smooth surface texture. In some embodiments, the surface texture of the N electrode 194 has an Ra that is less than about 58, less than about 55, or less than about 52. The surface texture of the N electrode 194 can be formed using any suitable method, for example, by mechanical processes (e.g., polishing) or by chemical processes (e.g., etching). In addition, the N electrode 194 can include other features that create a more hydrophilic surface on some or all of the N electrode 194 when compared to the surface of the V electrode 192.

In some embodiments, the size of the electrodes 192, 194 are about the same. In some embodiments, the diameter of the electrodes 186, 188 is about 0.50 millimeters (mm) to about 1.0 mm. In some embodiments, the length of the electrodes 192, 194 is about 0.50 mm to about 1.5 mm. In some embodiments, the exposed surface area of the electrodes 192, 194 is about 1.0 mm² to about 3.5 mm² each. Due to the corrugations of the V electrode 192, the V electrode 192 may be shorter than the N electrode 194 so that the electrodes 192, 194 have approximately the same exposed surface areas.

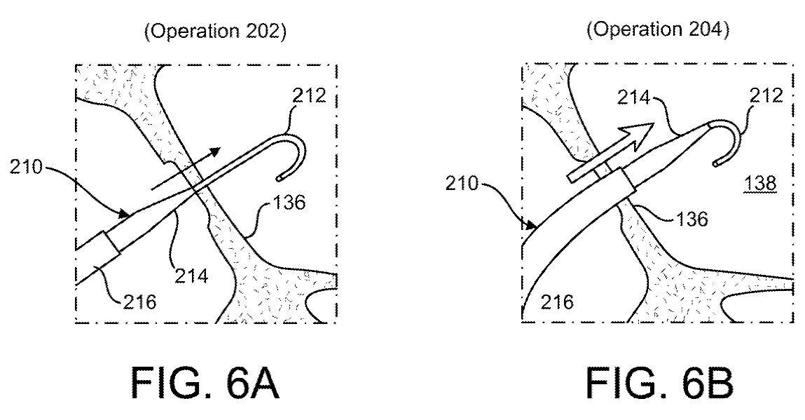

FIG. 5 shows a method 200 of crossing the atrial septum 136 using the crossing system 210 which can be the same as or similar to the crossing system 106 (shown in FIG. 2). FIGS. 6A and 6B show the operations of crossing the atrial septum 136. The distal tip of the crossing system 210 can have any of the embodiments of the crossing device of the present disclosure (e.g., crossing device 154, 178, 184, or 190). FIGS. 5 and 6A and 6B will now be discussed in conjunction with one another, and each operation of the method 200 is illustrated by a corresponding one of FIGS. 6A and 6B.

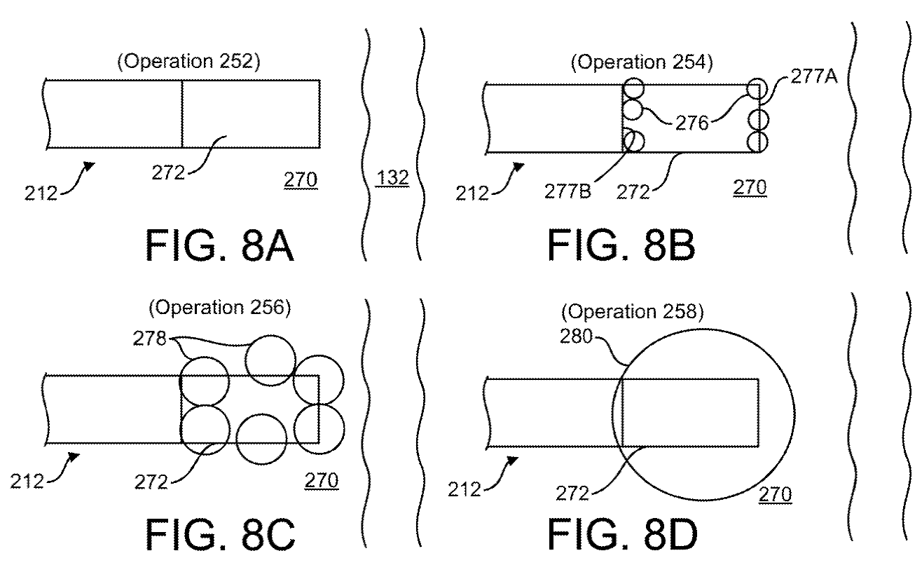

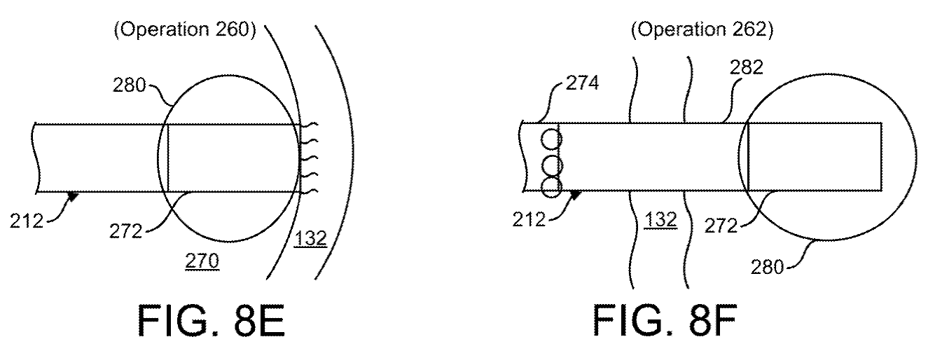

In the illustrated embodiment, the method 200 begins with a crossing system 210 being positioned near the atrial septum 136. At operation 202, as shown in FIG. 6A, the crossing device 212 is energized by the electrosurgical generator 104, and the physician applies distally-oriented force to the crossing device 212 (as indicated by the arrow). Thus, the crossing device 212 punctures through the atrial septum 136, and the crossing device 212 is deenergized. As shown in FIG. 8A, the crossing device 212 has a J-shape that is elastically deformed to be straight when the crossing device 212 is in the dilator 214, so the crossing device 212 returns to the J-shape when the crossing device 212 exits the dilator 214.

At operation 204, as shown in FIG. 6B, the physician applies distally-oriented force to the dilator 214 and to the sheath 216 (as indicated by the arrow). Thus, the dilator 214 and the sheath 216 follow the crossing device 212 through the atrial septum 136. As the dilator 214 follows the crossing device 212, the puncture in the atrial septum 136 is gently stretched so that the sheath 216 can enter the left atrium 138. Once the sheath 216 is positioned in the left atrium 138, one or more of the crossing device 212, the dilator 214, and the sheath 216 can be withdrawn and replaced with another component, such as, for example, a left atrial appendage closure (LAAC) implant device (not shown).

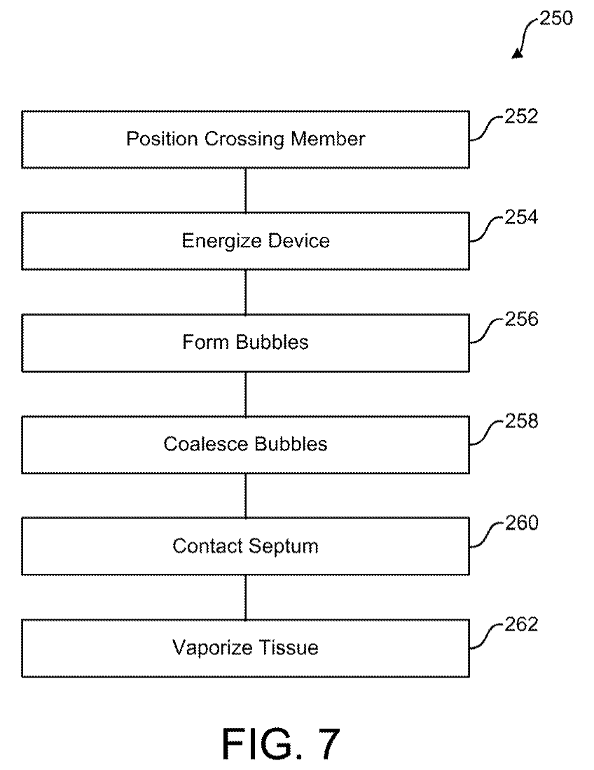

FIG. 7 shows a method 250 of puncturing the atrial septum 136. FIGS. 8A-8F show the operations of puncturing the atrial septum 136 using the crossing device 212. FIGS. 7 and 8A-8F will now be discussed in conjunction with one another, and each operation of the method 250 is illustrated by a corresponding one of FIGS. 8A-8F.

In the illustrated embodiment, the method 250 begins with operation 252. As shown in FIG. 8A, the crossing device 212 is positioned near the atrial septum 136 and surrounded by the blood 270.

At operation 254, as shown in FIG. 8B, the crossing device 212 is energized by the electrosurgical generator 104 (shown in FIG. 1), so RF energy is delivered to the V electrode 272 and the N electrode 274 (shown in FIG. 8F). The V electrode 272 can be any of the embodiments of a V electrode of the present disclosure (e.g., V electrode 156, 180, 186, or 192), and the N electrode 274 can be any of the embodiments of an N electrode 274 of the present disclosure (e.g., N electrode 158, 182, 188, or 194). Energizing the V electrode 272 passes RF energy into the blood 270 that surrounds the V electrode 272 since the blood 270 is electrically conductive. This locally gasifies the blood 270 and forms small gaseous bubbles 276 that stick to the V electrode 272 due to the configuration of the V electrode 272, especially around the major nucleation sites. More specifically, these major sites are along the distal and proximal circular edges 277A-277B of the electrode 272 (i.e., the circumferences of the distal and proximal ends of the V electrode 272, respectively). In addition, the N electrode 274 may also form such small gaseous bubbles (not shown) at its major nucleation sites (i.e., the circumferences of the distal and proximal ends of the N electrode 274).

At operation 256, as shown in FIG. 8C, the crossing device 212 is still energized and the V electrode 272 continues to generate heat and locally gasify the blood 270. The continued gasification rapidly increases the size of the small gaseous bubbles 276 (shown in FIG. 8B) to medium gaseous bubbles 278 due to the configuration differences between the V electrode 272 and the N electrode 274. For example, in some embodiments where the difference is based on surface texture, there are new small gaseous bubbles (not shown) that are grown on exterior areas of the V electrode 272 that are spaced apart from the major nucleation sites. These new small gaseous bubbles grow on the many minor nucleation sites along the surface of the electrode 272 that are provided by the rough and/or grooved surface texture of the V electrode 272, and said small gaseous bubbles also grow into the medium gaseous bubbles 278. Furthermore, the medium gaseous bubbles 278 are in a Wenzel state on the V electrode 272. The Wenzel state increases adhesion energy compared to the surface of the N electrode 274 that is substantially smooth, meaning that the medium gaseous bubbles 278 adhere more readily to the rough and/or grooved surface texture of the V electrode 272 than they would to the N electrode 274.

In contrast, the N electrode 274 does not have a configuration that locally concentrates current density as the V electrode 272 does. In some embodiments where the configuration difference is based on surface texture, along the length of the N electrode 274, there is no increased local heating, no minor nucleation sites (like the V electrode 272 has), no formation of additional small gaseous bubbles, and no growth of any small gaseous bubbles that were formed at operation 254. Furthermore, the smooth surface of the N electrode 274 does not have features that allow for small or medium gaseous bubbles to be in a Wenzel state on the N electrode 274. This means that any small gaseous bubbles that were formed on the N electrode 274 at the operation 254 would not be retained on the N electrode 274 if they grow at the operation 256.

At operation 258, as shown in FIG. 8D, the crossing device 212 is still energized and the electrode 272 continues to locally gasify the blood 270. The medium gaseous bubbles 278 (shown in FIG. 8C) grow and are retained on the V electrode 272 and eventually coalesce into a gaseous insulation layer 280. The gaseous insulation layer 280 is a single large gaseous bubble that surrounds substantially the entirety of the V electrode 272. While voltage is still being applied to the electrode 272 at the operation 258, the current flow through the V electrode 272 is greatly reduced compared to that of operations 254 and 256 since the V electrode 272 is electrically insulated from the blood 270. The great reduction in current flow means that less heat is produced, and less (if any) of the blood 270 is being locally gasified. Thus, the gaseous insulation layer 280 can maintain its size and adherence to the V electrode 272.

In contrast, the N electrode 274 would still only have a small number (if any) of small gaseous bubbles at its major nucleation sites. The N electrode 274 is unable to grow and retain gaseous bubbles, so there are never enough gaseous bubbles to coalesce and form a gaseous insulation layer around the N electrode 274. Furthermore, the number of small gaseous bubbles on the N electrode 274 may remain constant or decrease at the operation 258, because there is less electrical current flow into the N electrode 274 once the gaseous insulation layer 280 is formed around the V electrode 272.

At operation 260, as shown in FIG. 8E, the crossing device 212 is advanced by the physician so that the distal end of the V electrode 272 contacts the atrial septum 136. The gaseous insulation layer 280 is deformed as the distance between the electrode 272 and the atrial septum 136 closes. Eventually, the thickness of the gaseous insulation layer 278 between the electrode 272 and the atrial septum 136 is no longer sufficient, which causes a local dielectric breakdown of the gaseous insulation layer 280. Thereby, the distal end of the V electrode 272 and the atrial septum 136 are electrically connected, and virtually all of the RF energy being transmitted to the V electrode 272 flows into the cells of the atrial septum 136 that are closest to the V electrode 272, vaporizing the cells.

This RF energy is then returned to the N electrode 274 through, for example, the blood 270. As there are minimal gaseous bubbles on the N electrode 274, the returning current is dispersed along most of the length of the N electrode 274. Thus, even if the N electrode 274 was to come into contact with tissue (e.g., the atrial septum 136), the N electrode 274 would not be able to vaporize said tissue. Instead, at most, the tissue cells being contacted by the N electrode 274 would be desiccated due to the low concentration of RF energy. Desiccation increases the electrical resistance of the affected tissue cells, because there are fewer polar water molecules in the cells to conduct electricity, so the result is that even more power flows into the blood 270 instead of the tissue.

At operation 262, as shown in FIG. 8F, the physician has maintained distally-oriented force on the crossing device 212 as the cells at the end of the V electrode 272 have been vaporized. As these cells are vaporized, the crossing device 212 penetrates through the atrial septum 136 with minimal tenting of the atrial septum 136 and with minimal jumping of the crossing device 212 upon breakthrough. The ease of the puncture means that the physician does not need to exert as much force on the crossing device 212 compared to a prior art crossing system that has inefficient or diffuse RF energy delivery (à la N electrode 274).

In some embodiments, the time to puncture the atrial septum 136 can be about 300 ms to about 400 ms. Some electrosurgical generators, however, will continue to deliver power to the V electrode 272 due to their programming and/or user selection/input. The total “on” time of such a system can be, for example, multiple seconds (s) (e.g., 2 s to 5 s), which means that the V electrode 272 is still powered well after the puncture has been completed. In such a scenario, the gaseous insulation layer 280 may have been wiped off of the V electrode 272 as it passed through the atrial septum 136. But the gaseous insulation layer 280 can reform in the left atrium, for example, in a similar manner to that of operations 254-258. Once the gaseous insulation layer 280 is reformed, the amount of current flowing through the V electrode 272 will again be reduced until cessation of power delivery by the electrosurgical generator 104 (shown in FIG. 1). On the other hand, a prior art electrode that does not retain its gaseous bubbles, after puncturing the atrial septum 136, would be generating and releasing gaseous bubbles in the left atrium 138 of the heart 130 (shown in FIG. 2). Such free-floating bubbles can be dangerous to the patient 102 (shown in FIG. 1) for a variety of reasons.

The components and configuration of the crossing device 212 and the creation of the gaseous insulation layer 280 around the V electrode 272 reduces the amount of electrical power that is flowed through the crossing device 212. Thus, less heat is generated at the electrodes 272, 274, so there is less heat being transferred to the electrically insulative components of the crossing device 212 (e.g., insulator 282). The reduction in heat allows for an electrically insulative material to be employed in said components that is less heat resistant than in prior art crossing devices.

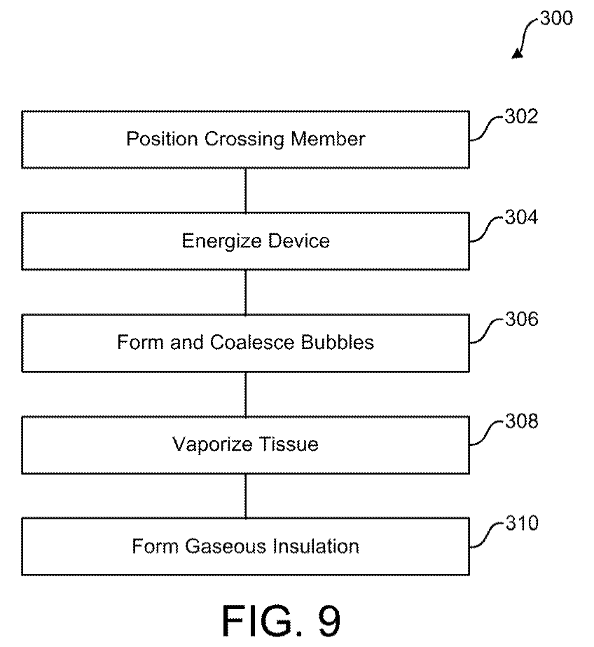

FIG. 9 shows a method 300 of puncturing the atrial septum 136. The method 300 is similar to that of the method 250 albeit with distinct differences. For example, the insulative gaseous layer is formed after the V electrode makes contact with the atrial septum instead of before.

In the illustrated embodiment, the method 300 begins with operation 302 wherein the crossing device is positioned in contact with the atrial septum.

At operation 304, the crossing device is energized, which also energizes the V and N electrodes. The electrodes can be any of the embodiments of the electrodes of the present disclosure, respectively. Energizing of the crossing device passes RF energy into the blood in and around the electrodes as well as into the atrial septum. Due to the diffuse distribution of RF energy, the cells in the atrial septum are not vaporized yet. The blood, however, is still locally gasified at the surfaces of the electrodes to form small gaseous bubbles at the major nucleation sites.

At operation 306, the crossing device is still energized and the V electrode continues to generate heat and locally gasify the blood. This gasification of the blood rapidly increases the size of the small gaseous bubbles, which are retained on the V electrode and eventually coalesce into a single large gaseous bubble. Such a large gaseous bubble is positioned between the V electrode and the blood, although not between the distal end of the V electrode and the atrial septum since those two were already in direct contact with one another.

At operation 308, the crossing device is still energized, and virtually all of the RF energy being transmitted to the V electrode now flows into the cells of the atrial septum. This energy vaporizes the cells of the atrial septum that are closest to (e.g., in direct contact with) the V electrode. As there are minimal gaseous bubbles on the N electrode, the returning current is dispersed along most of the length of the N electrode.

At operation 310, the physician has maintained distally-oriented force on the crossing device as the cells at the end of the V electrode have been vaporized. This vaporization allows the crossing device to puncture through the atrial septum. In doing so, a portion of the large gaseous bubble that existed outside of the V electrode may have been wiped off of the electrode as it passed through the atrial septum. But a gaseous insulation layer can be formed on the V electrode in the left atrium, for example, in a similar manner to that of operation 262 of the method 250 (shown in FIG. 7). Once the gaseous insulation layer is formed, the amount of current flowing through the V electrode will be greatly reduced until cessation of power delivery by the electrosurgical generator 104 (shown in FIG. 1). In addition, if the N electrode contacts the atrial septum at the operation 310, the atrial septum will not be further vaporized by the N electrode due to its insufficient and diffuse electrical current density.

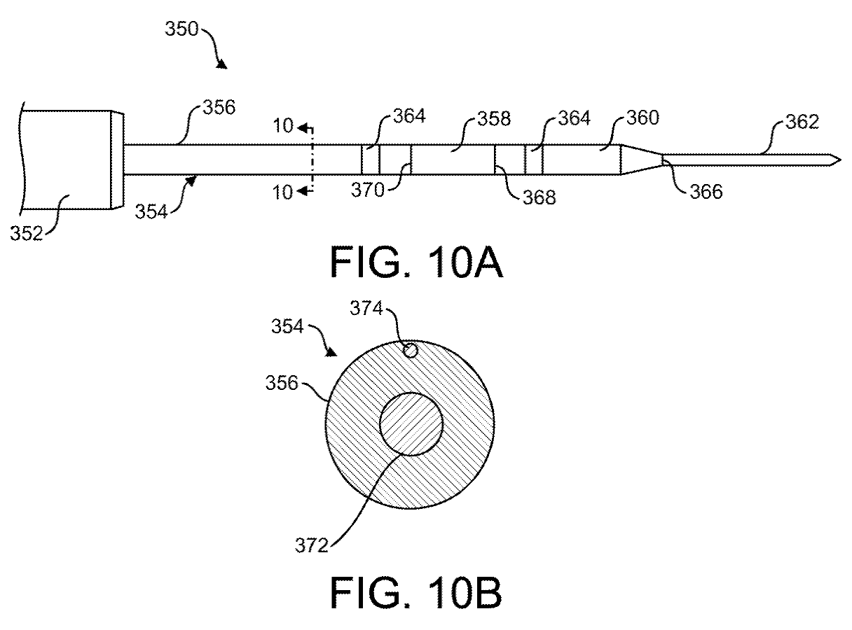

FIG. 10A shows a distal end of a dissecting system 350. In the illustrated embodiment, the dissecting system 350 includes a sheath 352 and a bipolar crossing device 354. In some embodiments, the dissecting system 350 has an overall length between about 55 cm and 300 cm. The sheath 352 is an elongate member with a central lumen (not shown) in which the crossing device 354 is slidably positioned. The central lumen diameter is similar to the outer diameter of the majority of the crossing device 354 (except for the distal tip). The sheath 352 can be a steerable sheath and/or have a fixed or adjustable curve at the distal end for positioning of the crossing device 354.

In the illustrated embodiment, the crossing device 354 is an elongate member with a shaft 356, a V electrode 358 adjacent to the shaft 356, a dilator 360 adjacent the V electrode 358, and an N electrode 362 at the distal end of the crossing device 354, adjacent to the dilator 360. The shaft 356 and the dilator 360 each include a radiopaque marker 364 that is positioned proximate to the V electrode 358. The N electrode 362 is a long and slender cylinder with a sharpened distal tip and terminates with a circular proximal edge 366 where the proximal end of the N electrode 362 and the distal end of the dilator 360 are coterminous. The V electrode 358 is a broader but shorter cylinder that terminates with distal and proximal circular ends 368, 370 where the V electrode 358 meets the shaft 356 and the dilator 360, respectively.

In the illustrated embodiment, shaft 356, the V electrode 358, and the dilator 360 (except for the distal end which tapers down to the size of the N electrode) all have substantially the same diameter, which is significantly wider than the diameter of the N electrode 362. In some embodiments, the diameter of the N electrode 362 is about 0.36 mm, and the diameter of the V electrode 358 is about 0.60 mm. In some embodiments, the surface areas of the V electrode 358 and the N electrode 362 are approximately the same (e.g., about 3.5 mm²). In some embodiments, the length of the N electrode 362 is about 3.0 mm whereas the length of the V electrode 358 is about 1.7 mm, so the N electrode 362 is at least about 1.5 times longer than the V electrode 358. In addition, the surface textures of the electrodes 358, 362 can be substantially the same (e.g., smooth), so, in comparison, the V electrode 358 is configured to form and retain insulative gaseous bubbles, whereas the N electrode 362 is not. Thus, the V electrode 358 is configured to vaporize tissue when energized in an electrically conductive fluid environment (e.g., when surrounded by blood), while the N electrode 362 is not.

FIG. 10B shows a cross-sectional view of the crossing device 354 as indicated by line 10-10 in FIG. 10A. In the illustrated embodiment, the shaft 356 includes a central lumen in which a return conductor 372 is positioned and an offset lumen in which an active conductor 374 is positioned. The active conductor 374 is electrically connected to the V electrode 358, and the return conductor 372 is electrically connected to the N electrode 362. The conductors 372, 374 and the electrodes 358, 362 comprise electrically conductive material, such as, for example, stainless steel, nitinol, platinum, gold, iridium, or combinations thereof. The return conductor 372 is electrically connected to one of the leads 112, 114 (shown in FIG. 1), and the active conductor 374 is electrically connected to the other one of the leads 112, 114.

In the illustrated embodiment, the V electrode 358 and the active conductor 374 are electrically insulated from the N electrode 362 and the return conductor 372 using electrically insulative layers and/or electrically insulative components (e.g., shaft 356 and/or dilator 360) that comprise electrically insulative material, such as, for example, parylene, polyimide, polyethylene terephthalate (PET), polyurethane, silicon dioxide (SiO₂), PTFE heat shrink, or combinations thereof).

The components and configuration of the dissecting system 350 allow for the N electrode 362 to mechanically puncture through tissue, such as, for example, a leaflet of the mitral valve 140 (shown in FIG. 2). Since the V electrode 358 is positioned proximal from the distal tip, the V electrode 358 can then be used to dissect or slice the tissue by moving the dissecting system 350 sideways (e.g., up and down, as depicted in FIGS. 10A and 10B). While the differentiating factor that makes the V electrode 358 vaporizing and the N electrode 362 non-vaporizing is the length difference between the two, in other embodiments, the differentiating factor could be surface texture, surface area, or a combination of factors balanced appropriately.

FIG. 11 shows an alternative distal end of a dissecting system 380. In the illustrated embodiment, the dissecting system 380 includes a sheath 382 and a bipolar crossing device 384. In some embodiments, the dissecting system 380 has an overall length between about 55 cm and about 300 cm. The sheath 382 is an elongate member with a central lumen (not shown) in which the crossing device 384 is slidably positioned. The central lumen diameter is similar to the outer diameter of the majority of the crossing device 384 (except for the distal tip). The sheath 382 can be a steerable sheath and/or have a fixed or adjustable curve at the distal end for positioning of the crossing device 384.

In the illustrated embodiment, the crossing device 384 is an elongate member with a shaft 386, a V electrode 388 adjacent to the shaft 386, a spacer 390 adjacent the V electrode 388, an N electrode 392 adjacent to the spacer 390, and a sharpened tip 394 at the distal end of the crossing device 384, adjacent to the N electrode 392. The shaft 386 and the dilator 360 each include a radiopaque marker 396 that is positioned proximate to the V electrode 388. The electrodes 388, 392 are cylinders that terminate with circular proximal and distal ends where the V electrode 388 meets the shaft 386 and the spacer 390 and where the N electrode 392 meets the spacer 390 and the tip 394, respectively.

In the illustrated embodiment, shaft 386, the V electrode 388, the spacer 390, and the tip 394 (except for the distal end which tapers to a point) all have substantially the same diameter. Thus, in some embodiments, the diameter of the crossing device 384 is about 1.8 mm. The lengths and surface areas of the V electrode 388 and the N electrode 392 are approximately the same (e.g., about 1.5 mm and 2.0 mm², respectively), so the surface texture of the V electrode 388 is significantly rougher than the surface texture of the N electrode 392 to differentiate the two. In some embodiments, the Ra surface texture of the V electrode 388 is between about 52 to about 64, between about 55 to about 61, or about 58; and the Ra surface texture of the N electrode 392 is less than about 58, less than about 55, or less than about 52.

In the illustrated embodiment, electrodes 388, 392 are electrically connected to the leads 112, 114 (shown in FIG. 1) via separate electrical conductors (not shown), respectively. In general, the configuration of the crossing device 384 allow for the electrodes 388, 392 to be electrically isolated from one another, so in some embodiments, the crossing device 384 has a similar internal configuration to that of crossing device 154 (shown in FIG. 3B) or crossing device 350 (shown in FIG. 10A). Unlike crossing devices 154, 350, both electrodes 388, 392 are positioned proximal from the distal end of the crossing device 384.

The components and configuration of the dissecting system 380 allow for the tip 394 to mechanically puncture through tissue, such as, for example, a leaflet of the mitral valve 140 (shown in FIG. 2). Since the V electrode 388 is positioned proximal from the distal tip, the V electrode 388 can then be used to dissect or slice the tissue by moving the dissecting system 380 sideways (e.g., up and down, as depicted in FIG. 11). While the differentiating factor that makes the V electrode 388 vaporizing and the N electrode 392 non-vaporizing is the surface texture difference between the two, in other embodiments, the differentiating factor could be length, surface area, or a combination of factors balanced appropriately.

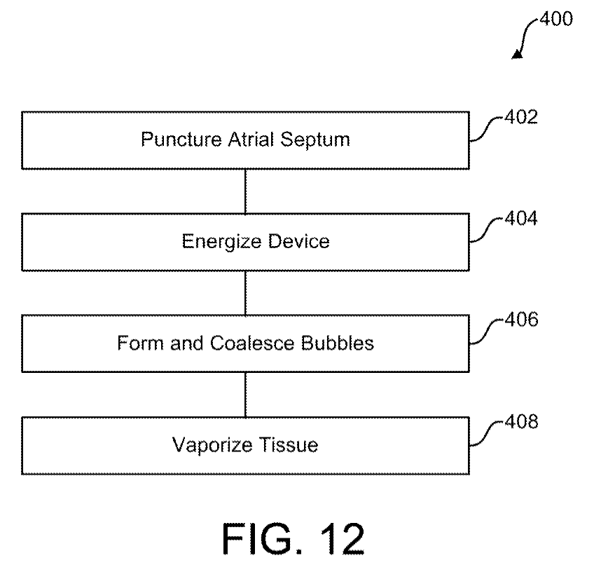

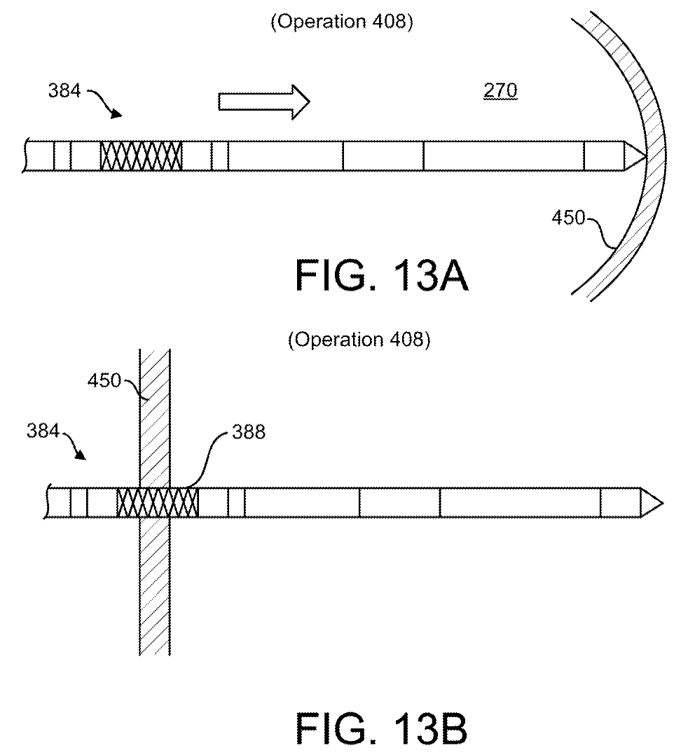

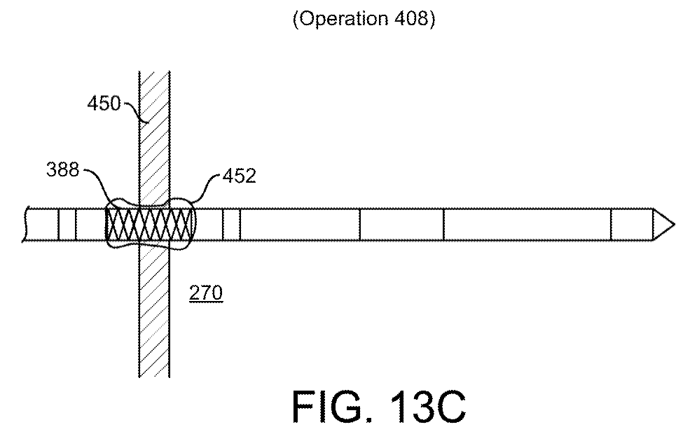

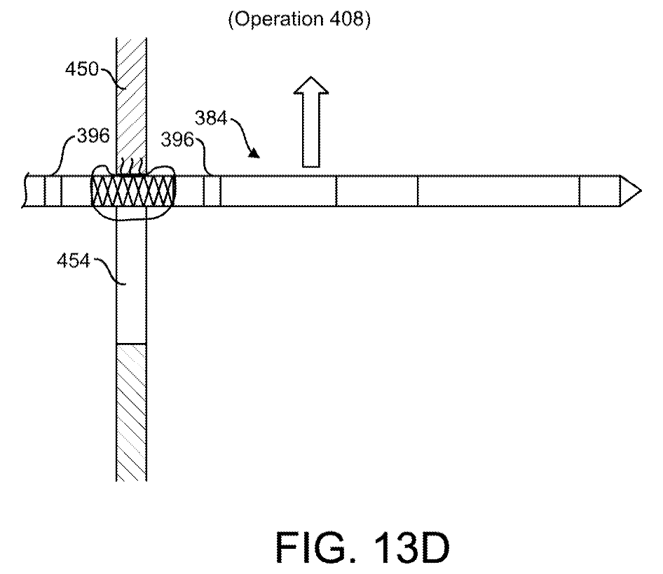

FIG. 12 shows a method 400 of puncturing and slicing tissue. FIG. 13A-13D show the operations of puncturing and slicing tissue using the crossing device 384, although the method 400 could be used with other configurations of crossing devices (e.g., crossing device 354, shown in FIG. 10A). FIGS. 12 and 13A-13D will now be discussed in conjunction with one another, and each operation of the method 400 is illustrated by a corresponding one of FIGS. 13A-13D.

In the illustrated embodiment, the method 400 begins with operation 402. As shown in FIG. 13A, the crossing device 384, which is surrounded by the blood 270, is forced in contact with the valve leaflet 450 by the physician (as indicated by the arrow in FIG. 13A).

At operation 404, as shown in FIG. 13B, the crossing device 384 mechanically punctures through the valve leaflet 450. In addition, the physician advances the crossing device 384 until the V electrode 388 is centered with respect to the valve leaflet 450.

At operation 406, as shown in FIG. 13C, the crossing device 384 is energized and the V electrode 388 locally gasifies the blood 270. The gasified blood forms a gaseous insulation layer 452 around the V electrode 388 (à la operations 304-306, shown in FIG. 9). In some embodiments, a portion of the circumference of the V electrode 388 is in direct contact with the valve leaflet 450. In such embodiments, the gaseous insulation layer 452 has two parts – one positioned on the entry side of the valve leaflet 450 and another on the exit side of the valve leaflet 450.

At operation 408, as shown in FIG. 13D, the crossing device is swept by the physician (as indicated by the arrow in FIG. 13D), so that the V electrode 388 vaporizes tissue cells in the valve leaflet 450. The result of the sideways slicing is that a slot 454 is formed in the valve leaflet 450. The operation 408 can be continued until the valve leaflet 450 has been shaped appropriately. During the operation 408, the gaseous insulation layer 452 promotes efficient tissue cell vaporization while minimizing heat generation in the crossing device 384. In addition, the differentiation (i.e., surface texture differences) between the V electrode 388 and the N electrode 392 ensures that the electrode that is positioned between the markers 396 is the electrode that is able to vaporize the tissue.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical system for removing tissue, the electrosurgical system comprising:
an electrosurgical generator configured to generate radiofrequency (RF) energy; and
a crossing device connected to the electrosurgical generator, the crossing device including a first electrode configured to deliver the RF energy to the tissue from the electrosurgical generator and a second electrode configured to return the RF energy to the electrosurgical generator;
wherein the first electrode is configured to form an electrically insulative gaseous layer when delivering the RF energy.

2. The electrosurgical system of claim 1, wherein the second electrode is configured to not form an electrically insulative gaseous layer when returning the RF energy.

3. The electrosurgical system of claim 1, wherein the first electrode has a smaller surface area than the second electrode.

4. The electrosurgical system of claim 1, wherein the first electrode has a rougher surface finish than the second electrode.

5. The electrosurgical system of claim 1, wherein the first electrode is shorter than the second electrode.

6. The electrosurgical system of claim 1, wherein the first electrode is positioned at a distal end of the crossing device, and the second electrode is positioned proximal from the first electrode.

7. The electrosurgical system of claim 1, wherein the second electrode is positioned at a distal end of the crossing device, and the first electrode is positioned proximal from the second electrode.

8. The electrosurgical system of claim 1, wherein the first electrode and the second electrode are positioned proximal from a distal end of the crossing device.

9. The electrosurgical system of claim 1, wherein the crossing device includes a central lumen.

10. An electrosurgical system for puncturing tissue, the electrosurgical system comprising:
an electrosurgical generator configured to generate radiofrequency (RF) energy; and
a crossing system comprising:
a steerable sheath;
a dilator slidably positioned in the steerable sheath; and
a crossing device connected to the electrosurgical generator, the crossing device including a vaporizing electrode positioned at a distal end of the crossing device and a no vaporizing electrode positioned proximal from the vaporizing electrode;
wherein the vaporizing electrode is configured to deliver the RF energy to the tissue from the electrosurgical generator and the non-vaporizing electrode is configured to return the RF energy to the electrosurgical generator.

11. The electrosurgical system of claim 10, wherein the vaporizing electrode is configured to form an electrically insulative gaseous layer when delivering the RF energy.

12. The electrosurgical system of claim 10, wherein the non-vaporizing electrode is configured to not form an electrically insulative gaseous layer when returning the RF energy.

13. The electrosurgical system of claim 10, wherein the first electrode has a smaller surface area than the second electrode.

14. The electrosurgical system of claim 10, wherein the first electrode has a rougher surface finish than the second electrode.

15. The electrosurgical system of claim 10, wherein the first electrode is shorter than the second electrode.

16. The electrosurgical system of claim 10, wherein the first electrode is positioned at a distal end of the crossing device, and the second electrode is positioned proximal from the first electrode.

17. The electrosurgical system of claim 10, wherein the second electrode is positioned at a distal end of the crossing device, and the first electrode is positioned proximal from the second electrode.

18. An electrosurgical system for slicing tissue, the electrosurgical system comprising:
an electrosurgical generator configured to generate radiofrequency (RF) energy; and
a crossing system comprising:
a steerable sheath;
a dilator slidably positioned in the steerable sheath; and
a crossing device slidably positioned in the dilator and electrically connected to the electrosurgical generator, the crossing device including a sharpened distal tip, a vaporizing electrode positioned proximal from the distal tip, and a non-vaporizing electrode positioned proximal from the distal tip;
wherein the vaporizing electrode is configured to deliver the RF energy to the tissue from the electrosurgical generator and the non-vaporizing electrode is configured to return the RF energy to the electrosurgical generator.

19. The electrosurgical system of claim 18, wherein the vaporizing electrode is configured to form an electrically insulative gaseous layer when delivering the RF energy.

20. The electrosurgical system of claim 18, wherein the non-vaporizing electrode is configured to not form an electrically insulative gaseous layer when returning the RF energy.
